(19) United States
(12) Patent Application Publication
(10) Pub. No.: US 2014/0171652 A1
(43) Pub. Date: Jun. 19, 2014

(54) PROCESSES FOR THE ISOLATION OF 4-AMINO-3-CHLORO-6-(4-CHLORO-2-FLUORO-3-METHOXYPHENYL)PYRIDINE-2-CARBOXYLIC ACID

(71) Applicants: Mark V. Emonds, Midland, MI (US); Robert C. Clouse, Midland, MI (US)

(72) Inventors: Mark V. Emonds, Midland, MI (US); Robert C. Clouse, Midland, MI (US)

(73) Assignee: DOW AGROSCIENCES LLC, Indianapolis, IN (US)

(21) Appl. No.: 14/103,894

(22) Filed: Dec. 12, 2013

Related U.S. Application Data
(60) Provisional application No. 61/736,820, filed on Dec. 13, 2012.

(51) Int. Cl. C07D 211/98

(52) U.S. Cl. CPC C07D 211/98
USPC 546/310

(57) ABSTRACT

Processes for the preparation and isolation of 4-amino-3-chloro-6-(4-chloro-2-fluoro-3-methoxyphenyl)pyridine-2-carboxylic acid.

### CROSS REFERENCE TO RELATED APPLICATIONS

This application claims the benefit of U.S. Provisional Patent Application Ser. No. 61/736,820 filed Dec. 13, 2012, the disclosure of which is expressly incorporated herein by reference.

### BACKGROUND

Provided herein are improved processes for the preparation and isolation of 4-amino-3-chloro-6-(4-chloro-2-fluoro-3-methoxyphenyl)pyridine-2-carboxylic acid.

U.S. Pat. No. 7,314,849 (B2) describes inter alia the preparation 4-amino-3-chloro-6-(poly-substitutedphenyl)pyridine-2-carboxylic acids and their use as herbicides, including 4-amino-3-chloro-6-(4-chloro-2-fluoro-3-methoxyphenyl)pyridine-2-carboxylic acid. The parent acids are conveniently prepared by the hydrolysis of either the corresponding esters or the corresponding protected N-acetylated esters. However, when the sodium salt of 4-amino-3-chloro-6-(4-chloro-2-fluoro-3-methoxyphenyl)pyridine-2-carboxylic acid resulting from hydrolysis is neutralized with an inorganic acid such as hydrochloric acid, a very fine precipitate is formed which is difficult to filter. While use of acetic acid improves upon this method, the precipitate is still difficult to process, giving both slurries that are difficult to agitate and crystals that are difficult to filter. In addition, use of acetic acid also can result in product crystals which contain occluded acetic acid, which cannot be removed by reslurrying the product.

It would be advantageous to have a method to neutralize the sodium salt of 4-amino-3-chloro-6-(4-chloro-2-fluoro-3-methoxyphenyl)pyridine-2-carboxylic acid that would improve the ability to process and handle the resulting 4-amino-3-chloro-6-(4-chloro-2-fluoro-3-methoxyphenyl)pyridine-2-carboxylic acid.

### SUMMARY

Provided herein are improved processes for the isolation of 4-amino-3-chloro-6-(4-chloro-2-fluoro-3-methoxyphenyl)pyridine-2-carboxylic acid which comprise (a) neutralizing an aqueous solution of an alkali metal or alkaline earth metal salt of 4-amino-3-chloro-6-(4-chloro-2-fluoro-3-methoxyphenyl)pyridine-2-carboxylic acid with an excess of 85-99 percent formic acid at a temperature from about 45 to about 90° C. to produce an aqueous mixture of 4-amino-3-chloro-6-(4-chloro-2-fluoro-3-methoxyphenyl)pyridine-2-carboxylic acid, (b) cooling the aqueous mixture of 4-amino-3-chloro-6-(4-chloro-2-fluoro-3-methoxyphenyl)pyridine-2-carboxylic acid to about 10 to about 25° C. to crystallize the 4-amino-3-chloro-6-(4-chloro-2-fluoro-3-methoxyphenyl)pyridine-2-carboxylic acid, and (c) collecting the crystalline 4-amino-3-chloro-6-(4-chloro-2-fluoro-3-methoxyphenyl)pyridine-2-carboxylic acid.

Provided herein are also improved processes for the preparation and isolation of 4-amino-3-chloro-6-(4-chloro-2-fluoro-3-methoxyphenyl)pyridine-2-carboxylic acid which comprise (a) contacting an ester of 4-amino-3-chloro-6-(4-chloro-2-fluoro-3-methoxyphenyl)pyridine-2-carboxylic acid of Formula I

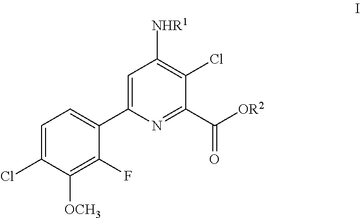

in which
R¹ represents H or C(O)CH₃, and
R² represents C₁-C₁₂ alkyl or an unsubstituted or substituted C₇-C₁₁ arylalkyl with an aqueous solution of an alkali metal or alkaline earth metal hydroxide in a C₁-C₄ alcohol at a temperature from about 45 to about 100° C. to produce an aqueous alcoholic solution of the alkali metal or alkaline earth metal salt of 4-amino-3-chloro-6-(4-chloro-2-fluoro-3-methoxyphenyl)pyridine-2-carboxylic acid, (b) optionally removing most of the C₁-C₄ alcohol from the aqueous alcoholic solution of the alkali metal or alkaline earth metal salt of 4-amino-3-chloro-6-(4-chloro-2-fluoro-3-methoxyphenyl)pyridine-2-carboxylic acid, (c) neutralizing the aqueous solution of the alkali metal or alkaline earth metal salt of 4-amino-3-chloro-6-(4-chloro-2-fluoro-3-methoxyphenyl)pyridine-2-carboxylic acid with an excess of 85-99 percent formic acid at a temperature from about 45 to about 90° C. to produce an aqueous mixture of 4-amino-3-chloro-6-(4-chloro-2-fluoro-3-methoxyphenyl)pyridine-2-carboxylic acid, (d) cooling the aqueous mixture of 4-amino-3-chloro-6-(4-chloro-2-fluoro-3-methoxyphenyl)pyridine-2-carboxylic acid to about 10 to about 25° C. to crystallize the 4-amino-3-chloro-6-(4-chloro-2-fluoro-3-methoxyphenyl)pyridine-2-carboxylic acid, and (e) collecting the crystalline 4-amino-3-chloro-6-(4-chloro-2-fluoro-3-methoxyphenyl)pyridine-2-carboxylic acid.

### DETAILED DESCRIPTION

The term alkyl and derivative terms such as alcohol, as used herein refer to straight chain or branched chain moieties. Typical C₁-C₄ alkyl groups are methyl, ethyl, propyl, 1-methylethyl, butyl, 1,1-dimethylethyl and 1-methylpropyl. Methyl and ethyl are often preferred.

The term “arylalkyl,” as used herein, refers to a phenyl substituted alkyl group having a total of 7 to 11 carbon atoms, such as benzyl (—CH₂C₆H₅), 2-methylnaphthyl (—CH₂C₁₀H₇) and 1- or 2-phenethyl (—CH₂CH₂C₆H₅ or —CH(CH₃)C₆H₅). The phenyl group may itself be unsubstituted or substituted with one or more substituents independently selected from halogen, nitro, cyano, C₁-C₆ alkyl, C₁-C₆ alkoxy, halogenated C₁-C₆ alkyl, halogenated C₁-C₆ alkoxy, C₁-C₆ alkylthio, C(O)OC₁-C₆ alkyl, or where two adjacent substituents are taken together as —O(CH₂)ₙO— wherein n=1 or 2, provided that the substituents are sterically compatible and the rules of chemical bonding and strain energy are satisfied.

Alkali metals and alkaline earth metals refer to members of groups 1 and 2 of the periodic table. Preferred alkali metal (group 1) hydroxides are sodium hydroxide and potassium hydroxide. Preferred alkaline earth metal (group 2) hydroxides are magnesium hydroxide and calcium hydroxide.

By neutralizing with formic acid, crystals of 4-amino-3-chloro-6-(4-chloro-2-fluoro-3-methoxyphenyl)pyridine-2-carboxylic acid that are easier to handle are obtained. In the improved process, an aqueous solution of an alkali metal or alkaline earth metal salt of 4-amino-3-chloro-6-(4-chloro-2-fluoro-3-methoxyphenyl)pyridine-2-carboxylic acid is neutralized with an excess of 85-99 percent formic acid at a temperature from about 45 to about 90° C. to produce an aqueous mixture of 4-amino-3-chloro-6-(4-chloro-2-fluoro-3-methoxyphenyl)pyridine-2-carboxylic acid. It is often most convenient to add the aqueous solution of an alkali metal or alkaline earth metal salt of 4-amino-3-chloro-6-(4-chloro-2-fluoro-3-methoxyphenyl)pyridine-2-carboxylic acid to a preheated large molar excess of formic acid (>10 times). While concentrations of formic acid from about 85-99 percent are acceptable, better crystalizations occur at higher concentrations of formic acid. Usually, the neutralized mixture is maintained in the temperature range from about 45 to about 90° C. for about 1 hour before cooling. At the lower end of the temperature range, crystal formation may initate before cooling. After cooling, the crystalline product can be collected by standard procedures such as filtration or centrifugation.

In some embodiments, the alkali metal or alkaline earth metal salt of 4-amino-3-chloro-6-(4-chloro-2-fluoro-3-methoxyphenyl)pyridine-2-carboxylic acid that is subsequently neutralized is prepared by the hydrolysis/deprotection of esters or protected N-acetylated esters of 4-amino-3-chloro-6-(4-chloro-2-fluoro-3-methoxyphenyl)pyridine-2-carboxylic acid. In the initial steps of this process, the ester or protected N-acetylated ester of 4-amino-3-chloro-6-(4-chloro-2-fluoro-3-methoxyphenyl)pyridine-2-carboxylic acid is contacted with an aqueous solution of an alkali metal or alkaline earth metal hydroxide in a C₁-C₄ alcohol at a temperature from about 45 to about 100° C. While only one equivalent of hydroxide is required for each ester and protecting group, it is preferable to employ an excess. Sodium and potassium hydroxide are the preferred alkali metal or alkaline earth metal hydroxides. Methanol is the preferred C₁-C₄ alcohol and the preferred temperature range for this alcohol is from about 45 to about 65° C.

Once the ester has been hydrolyzed and the protecting group removed, most of the C₁-C₄ alcohol may be removed from the aqueous alcoholic solution of the alkali metal or alkaline earth metal salt of 4-amino-3-chloro-6-(4-chloro-2-fluoro-3-methoxyphenyl)-pyridine-2-carboxylic acid. The C₁-C₄ alcohol is conveniently removed under reduced pressure, leaving a solution of the alkali metal or alkaline earth metal salt of 4-amino-3-chloro-6-(4-chloro-2-fluoro-3-methoxyphenyl)pyridine-2-carboxylic acid in predominantly water. Depending on the amount of C₁-C₄ alcohol that is employed, this step may be omitted. It is the aqueous solution containing small-to-negligible amounts of alcohol that is then neutralized and cooled and from which 4-amino-3-chloro-6-(4-chloro-2-fluoro-3-methoxyphenyl)pyridine-2-carboxylic acid is obtained as crystals as described previously.

The described embodiments and following examples are for illustrative purposes and are not intended to limit the scope of the claims. Other modifications, uses, or combinations with respect to the compositions described herein will be apparent to a person of ordinary skill in the art without departing from the spirit and scope of the claimed subject matter.

### EXAMPLES

### Example 1

To a 250 milliliter (mL) flask equipped with a magnetic stirrer, condenser, and nitrogen (N₂) bubbler were added methyl 4-(acetylamino)-3-chloro-6-(4-chloro-2-fluoro-3-methoxyphenyl)pyridine-2-carboxylate (5.0 grams (g))_, methanol (MeOH; 16 mL), and water (4 mL). A solution of 50% sodium hydroxide (NaOH; 2.3 g) in water (4 mL) was added, and the mixture was heated to reflux for 2-2.5 hours (h). The mixture was allowed to cool to 20-25° C. and filtered using Whatman #50 paper. The filtrate was concentrated on a rotary evaporator at about 25° C. to give a crude aqueous solution of 15.2-15.7 g. To a 250 mL flask equipped with a mechanical stirrer, condenser, and N₂ bubbler was added 98% formic acid (40 mL). The acid was heated to about 50° C., and the aqueous solution was added to the formic acid over 1-2 minutes (min) at 50° C. After several min, the product began to crystallize from solution. The resulting slurry was maintained at 50° C. for about 1 h and then allowed to cool to 20-21° C. over 1-1.5 h. Water (20 mL) was added, and the slurry was stirred for 1-2 h at 20-21° C. The slurry was filtered using Whatman #50 paper, and the wet cake was washed twice with water (2×30 mL). After drying, 4-amino-3-chloro-6-(4-chloro-2-fluoro-3-methoxyphenyl)pyridine-2-carboxylic acid was obtained as a white solid (4.0 g, 93.5% yield).

### Example 2

Organically wet methyl 4-amino-3-chloro-6-(4-chloro-2-fluoro-3-methoxyphenyl)-pyridine-2-carboxylate solid (253.4 pounds (lb), containing 5.8% toluene and isooctane and 231.7 lb of actual methyl 4-amino-3-chloro-6-(4-chloro-2-fluoro-3-methoxyphenyl)pyridine-2-carboxylate) was loaded into a 300-gallon (gal) metal reaction vessel and the vessel was purged with nitrogen. Aqueous NaOH (221 lb of a 24.4% NaOH solution in water) was pumped into the reactor, followed by water (199 lb). Methanol (52 lb) was pumped into the reactor, and the mixture was stirred and heated to 67-68° C. for 3.25 h. The reactor contents were then cooled to 25° C. and filtered through a Celite® bed (10 lb) on a centrifuge. The filtered solution was transferred as it was filtered into a stirred 500-gal stainless steel reaction vessel containing 98% formic acid (2266 lb) at 50° C. The neutralized mixture was slowly cooled to 20° C. at a rate of 10° C./h to precipitate the product. When the mixture had cooled to 20° C., additional water (96 gal) was added. The precipitated 4-amino-3-chloro-6-(4-chloro-2-fluoro-3-methoxyphenyl)pyridine-2-carboxylic acid was collected via centrifugation and washed with water. A total crude yield of 539.0 lb of wet product was obtained. This product was loaded into the 500-gal stainless steel reactor and suspended in water (450 gal). The mixture was stirred for 3 h, and then the solids were collected via centrifugation and washed with water (1000 lb). A wet cake of 4-amino-3-chloro-6-(4-chloro-2-fluoro-3-methoxyphenyl)pyridine-2-carboxylic acid (513.5 lb) was collected, which contained 176.45 lb of product on a dry basis (87.7% yield, with a dry basis purity of 98.6%). Residual formate levels were <0.07% by ¹H NMR, calculated as formic acid.

### Comparative Example 3 (Acidification with Acetic Acid)

Methyl 4-amino-3-chloro-6-(4-chloro-2-fluoro-3-methoxyphenyl)pyridine-2-carboxylate (1.24 kilograms (kg), 3.2 moles (mol)) was loaded into a 12-liter (L) three-necked flask equipped with a heating mantle and suspended in a mixture of water (1.00 kg) and methanol (3.14 kg). Diluted aqueous sodium hydroxide (NaOH; 2.2 equivalents (equiv); 0.99 kg water +0.57 kg 50% NaOH) was then added, and the mixture was stirred and heated to reflux (64.7° C.). The reaction was sampled and analyzed by high-performance liquid chromatography (HPLC) to ensure complete hydrolysis. The mixture was then filtered hot through an inline filter equipped with a glass frit on which a pad of Celite® was laid. The purpose of this filtration was to remove small quantities of insoluble inorganic salts that were present in the methyl 4-amino-3-chloro-6-(4-chloro-2-fluoro-3-methoxyphenyl)pyridine-2-carboxylate intermediate. The filtered solution of the sodium 4-amino-3-chloro-6-(4-chloro-2-fluoro-3-methoxyphenyl)pyridine-2-carboxylate was concentrated on a rotary evaporator to remove the methanol. The aqueous solution was then transferred to a 22-L glass reactor. Glacial acetic acid (54.1 equiv, 10.42 kg) was added with stirring to precipitate the product. The resulting slurry was warmed to 50° C. for about an hour and then cooled. The solids were recovered by filtration using a Buchner-type filter crock with Whatman #50 filter paper. The solids were washed with water, then returned to the 22-L glass reactor. Previous batches had shown residual levels of acetic acid in the solid ranging from 0.3-47.0% at this stage. Water (9.5 kg) was added and the resulting slurry was stirred for 1.5 h, and the solids were again recovered by filtration as described previously. After recovery, the solids were air dried, and then dried in a vacuum oven. The product was analyzed by HPLC and ¹H NMR spectroscopy and was found to contain 1.54% acetic acid. The solids were returned to the 22-L reactor and again slurried in water, filtered, and dried. After this treatment, the acetic acid level was reduced to 0.87% by weight. A third slurry reduced the acetic acid level to 0.84% by weight. The final yield of product was 0.906 kg of with an assay of 97.59%, and contained 0.84% acetic acid and 0.34% water. The yield was 83.44% of the theoretical amount. Another subsequent batch run in this manner maintained an acetic acid level of 2.25% after being slurried in water two times.

What is claimed is:

1. A process for the isolation of 4-amino-3-chloro-6-(4-chloro-2-fluoro-3-methoxyphenyl)pyridine-2-carboxylic acid which comprises:
(a) neutralizing an aqueous solution of an alkali metal or alkaline earth metal salt of 4-amino-3-chloro-6-(4-chloro-2-fluoro-3-methoxyphenyl)pyridine-2-carboxylic acid with an excess of 85-99 percent formic acid at a temperature from about 45 to about 90° C. to produce an aqueous mixture of 4-amino-3-chloro-6-(4-chloro-2-fluoro-3-methoxyphenyl)pyridine-2-carboxylic acid;
(b) cooling the aqueous mixture of 4-amino-3-chloro-6-(4-chloro-2-fluoro-3-methoxyphenyl)pyridine-2-carboxylic acid to about 10 to about 25° C. to crystallize the 4-amino-3-chloro-6-(4-chloro-2-fluoro-3-methoxyphenyl)pyridine-2-carboxylic acid, and
(c) collecting the crystalline 4-amino-3-chloro-6-(4-chloro-2-fluoro-3-methoxyphenyl)-pyridine-2-carboxylic acid.

2. A process for the preparation and isolation of 4-amino-3-chloro-6-(4-chloro-2-fluoro-3-methoxyphenyl)pyridine-2-carboxylic acid which comprises:
(a) contacting an ester of 4-amino-3-chloro-6-(4-chloro-2-fluoro-3-methoxyphenyl)pyridine-2-carboxylic acid of Formula I 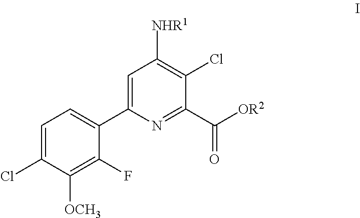
in which
R¹ represents H or C(O)CH₃, and
R² represents C₁-C₁₂ alkyl or an unsubstituted or substituted C₇-C₁₁ arylalkyl
with an aqueous solution of an alkali metal or alkaline earth metal hydroxide in a C₁-C₄ alcohol at a temperature from about 45 to about 100° C. to produce an aqueous alcoholic solution of the alkali metal or alkaline earth metal salt of 4-amino-3-chloro-6-(4-chloro-2-fluoro-3-methoxyphenyl)pyridine-2-carboxylic acid,
(b) optionally removing most of the C₁-C₄ alcohol from the aqueous alcoholic solution of the alkali metal or alkaline earth metal salt of 4-amino-3-chloro-6-(4-chloro-2-fluoro-3-methoxyphenyl)pyridine-2-carboxylic acid,
(c) neutralizing the aqueous solution of the alkali metal or alkaline earth metal salt of 4-amino-3-chloro-6-(4-chloro-2-fluoro-3-methoxyphenyl)pyridine-2-carboxylic acid with an excess of 85-99 percent formic acid at a temperature from about 45 to about 90° C. to produce an aqueous mixture of 4-amino-3-chloro-6-(4-chloro-2-fluoro-3-methoxyphenyl)pyridine-2-carboxylic acid,
(d) cooling the aqueous mixture of 4-amino-3-chloro-6-(4-chloro-2-fluoro-3-methoxyphenyl)pyridine-2-carboxylic acid to about 10 to about 25° C. to crystallize the 4-amino-3-chloro-6-(4-chloro-2-fluoro-3-methoxyphenyl)pyridine-2-carboxylic acid, and
(e) collecting the crystalline 4-amino-3-chloro-6-(4-chloro-2-fluoro-3-methoxyphenyl)pyridine-2-carboxylic acid.
